(19) United States
(12) Patent Application Publication
(10) Pub. No.: US 2017/0202499 A1
(43) Pub. Date: Jul. 20, 2017

(54) CANNULA ASSEMBLY

(71) Applicant: Shalom MANOVA, Savion (IL)

(72) Inventor: Patrick COHEN, Maytar (IL)

(21) Appl. No.: 15/422,106

(22) Filed: Feb. 1, 2017

Related U.S. Application Data
(63) Continuation of application No. PCT/IL2015/050794, filed on Aug. 2, 2015.
Application No. PCT/IL2015/050794 is a continuation-in-part of application No. 14/449,515, filed on Aug. 1, 2014.

(51) Int. Cl. A61B 5/153; A61M 5/158

(52) U.S. Cl. CPC A61B 5/153; A61M 5/158

(57) ABSTRACT

This disclosure provides a cannula assembly with a flexible cannula that can be used for blood withdrawal or delivery of fluid that utilizes a main cannula that fixed with its end in a blood vessel that uses the cannula as a passageway into the blood vessel.

### TECHNOLOGICAL FIELD

This disclosure relates to medical devices, and more specifically, to cannula assemblies for transferring a fluid into or out of a body cavity, such as a blood vessel.

### BACKGROUND

Many medical procedures require access to a body cavity, such as a blood vessel, in order to deliver substances to the cavity or to withdraw fluids from the cavity. For example, a vein may be accessed in order to administer fluids and drugs, or for drawing blood for analyses. Accessing a body cavity, such as a blood vessel, often involves introducing a catheter onto a cannula and pushing the cannula tip through the skin and into the body until the tip of the cannula and the catheter are positioned inside the lumen of the vein or artery. The cannula or hollow needle is then removed while leaving the flexible catheter in place, which thus forms a conduit for the infusion or withdrawal of fluids between the vessel and the exterior of the body. When multiple transfers of fluid into or out of a vessel are required, the catheter can be left in place in order to reduce the need to reinsert the cannula or needle into the body, which is inconvenient and often painful for the patient.

One way to avoid repeated insertion of a needle into the vein is to use a venous or arterial cannula, which is usually a thin tube, the distal end of which is inserted through the skin into a blood vessel and maintained indwelling in the blood vessel with the proximal end of the tube being accessible on the skin surface. A trocar is used to puncture the vein and insert the silicon cannula that remains in the vein. Common complications associated with cannulation of a blood vessel include hematoma (accumulation and clotting of blood due to failure to puncture the blood vessel when the cannula is inserted), infiltration (delivery of an infusate to subcutaneous tissue instead of the vein), embolism (caused by air, a thrombus, or fragment of a catheter breaking off and entering the venous system), or phlebitis (inflammation of the vein resulting from mechanical or chemical irritation or from an infection). These complications are often more serious in children and elderly patients repeatedly exposed to cannula insertion and withdrawal.

In addition, in order to withdraw blood and because one cannot readily use a cannula that is already in a vein, there is often a need to re-puncture the patient's vein, often once or twice daily. Repeated punctures, particularly in the elderly and in children, may cause hematomas or infection and may also cause the veins to collapse, making it difficult to find a suitable vein from which to withdraw blood.

### GENERAL DESCRIPTION

This disclosure provides a cannula assembly with a flexible cannula that can be used for withdrawal or delivery of fluid (e.g. blood) such that the flexible cannula utilizes a main cannula, which is fixed with its end into a body cavity, as a passageway for the flexible cannula into a body cavity, e.g. a blood vessel. More particularly, the present disclosure provides a cannula assembly that includes an additional element over existing cannula assemblies. This element is a flexible cannula, referred to below as “second cannula” that is configured for insertion through a main cannula, referred to below as “first cannula”.

In operation, the main cannula is operated, by the use of a trocar, as further detailed below, such that the open end at its distal portion comes to be within a blood vessel. The insertion of the flexible cannula through the main cannula then brings the end of the flexible cannula to be within the same blood vessel and allow blood withdrawal or delivery of a fluid, e.g. a liquid medicament, therethrough. Typically, the length of the flexible cannula is such so its distal end extends beyond that of the distal end of the main cannula by a length chosen to be sufficient to avoid collapse of a patient's blood vessel in which the cannulae (first and second cannula) are inserted.

In this disclosure the term “distal” refers to that end of the device which is inserted into the patient's body (e.g. penetrated through the skin and into a blood vessel) while the term “proximal” is that end which in use is in proximity to the caregiver.

The present disclosure provides a cannula assembly comprising a first cannula device, a trocar element and at least one second flexible cannula. The first cannula device has a first length and first inner diameter and has at least a flexible distal portion. The trocar element has a second length and second external diameter and has a sharp distal end capable of puncturing through skin and one or more blood vessels of a patient. The trocar is sized such so as that the second diameter permits it to be inserted within the first cannula and has a length so once inserted its distal end can extend beyond the flexible distal portion of the first cannula. The second, outer diameter of the trocar is typically such so that it fits snuggly within the lumen of the first cannula with the outer walls of the trocar being proximal, e.g. touching, the inner walls of the first cannula. The flexible second cannula is configured for insertion through the first cannula device, each second flexible cannula sized to extend beyond the distal portion of the first cannula for a predetermined length within the blood vessel of the patient. The predetermined length of the second cannula is chosen to be sufficient to avoid collapse of a patient's blood vessel. The proximal end of the second cannula is configured for engagement with a blood withdrawal or a fluid delivery device; for example with a syringe, a blood collection device, e.g. blood collection tube with pre-formed vacuum or an intravenous solution adapter.

This disclosure further provides a method of inserting a cannula into a blood vessel to deliver a fluid such as infusion to a blood vessel or other body cavity or withdraw a fluid from a blood vessel or other body cavity. The method comprises (i) providing a cannula assembly of the kind having described above; (ii) inserting the first cannula and the trocar within it into a blood vessel of a patient by puncturing through skin into the blood vessel of the patient; (iii) withdrawing trocar from the first cannula; and (iv) inserting the second cannula into the first cannula such that the distal end of the second cannula extends a distance in the blood vessel of the patient beyond the distal end of the first cannula, e.g. chosen to be sufficient to avoid collapse of a patient's blood vessel.

The trocar may be inserted into the first cannula such that the sharp distal tip of the trocar extends beyond the distal portion of the first cannula.

In line with acceptable medical practice the first cannula remains in place after insertion. The second cannula (typically a fresh and sterile one) may then be inserted each time another blood sample is needed or desired. This saves the patient from multiple punctures and the system, may be sized for use with adult, pediatric, neonatal or elderly patients.

Also provided by this disclosure is a flexible cannula configured to serve as said second cannula in the above assembly.

The assembly of this disclosure may be used, for example, for collecting a blood sample from a blood vessel or infusing a liquid such as an intravenous, e.g. medicated, fluid into a blood vessel. The assembly of this disclosure is particularly suited when repeated withdrawals blood or delivery of fluids are to be performed.

### BRIEF DESCRIPTION OF THE DRAWINGS

In order to better understand this disclosure and to see how it may be carried out in practice, embodiments will now be described, by way of non-limiting example only, with reference to the accompanying drawings, in which:

FIG. 1 is a schematic representation of a cannula assembly for withdrawing fluid from a body cavity or delivering fluid to a body cavity, in accordance with an embodiment of this disclosure;

FIG. 2 is a schematic representation of the trocar of the cannula assembly of FIG. 1 inserted into the first cannula device;

FIGS. 3A and 3B are schematic representations showing an exemplary use of the cannula assembly of FIG. 1, demonstrating removal or delivery of fluid (such as blood) from a blood vessel.

### DETAILED DESCRIPTION OF EMBODIMENTS

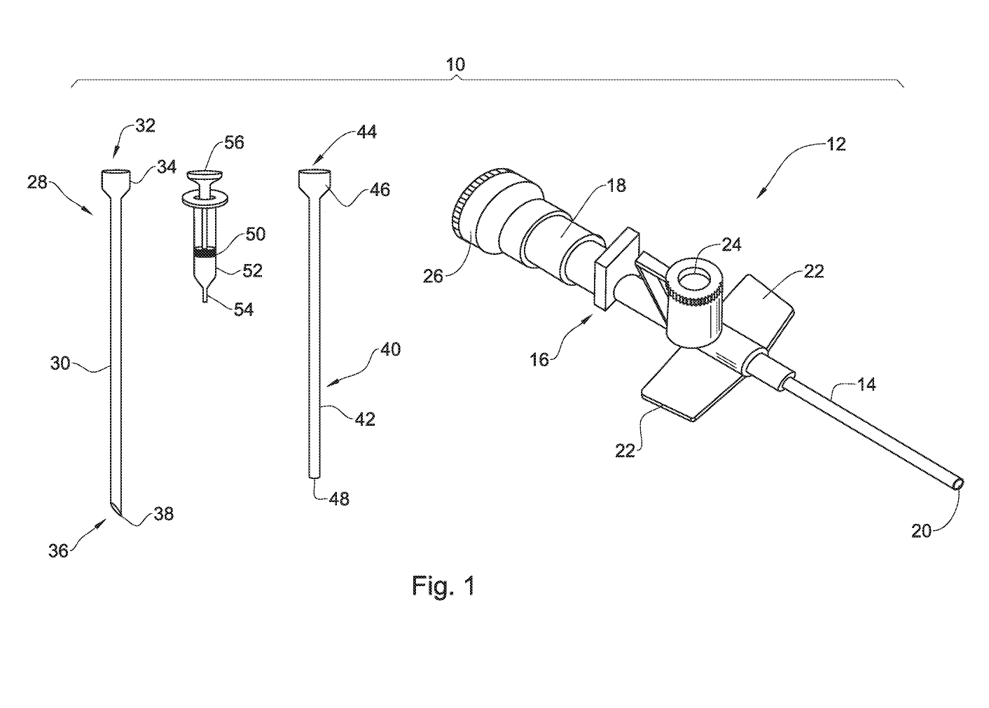

FIG. 1 shows a cannula assembly 10 for transferring a fluid into or out of a body cavity such as an artery, vein, or other vessel (not shown), in accordance with an embodiment of this disclosure. The cannula assembly 10 with comprises a first cannula device 12 having a flexible distal portion 14 mounted onto a holder 16. The first cannula device 12 also has a flashback chamber 18 that is used to determine when the distal end 20 of the first cannula device 12 is properly positioned inside a vessel as indicated by the presence of fluid, such as blood, in the flashback chamber 18, as explained below. Wings 22 on the holder 16 are used to assist in inserting the first cannula device 12 into a blood vessel and can then be used to fix the holder 16 to the skin using, for example, an adhesive tape (not shown). Fluids can enter or leave the first cannula device 12 either via a side port 24 or through a female Luer fitting 26, when a syringe or tubing (not shown) is attached to the female Luer fitting 26.

The cannula assembly 10 further comprises a trocar 28 having a slender, rigid, hollow tubular needle portion 30, a proximal end 32 that may be provided with a female Luer fitting 34 and a distal end 36 terminating in a sharp pointed tip 38 suitable for puncturing skin and blood vessels.

The cannula assembly 10 also comprises one or more flexible second cannula 40 having a flexible tubular portion 42, a proximal end 44 provided with a female Luer fitting 46, and a distal open end 48. The cannula assembly 10 may also comprise a syringe 50 (shown out of scale of the other system elements) having a barrel 52 terminating in a male Luer fitting 54 configured to mate with the female Luer fitting 46 on the flexible second cannula 40, and a plunger 56. The flexible cannula 40 may be integrated with the syringe 50.

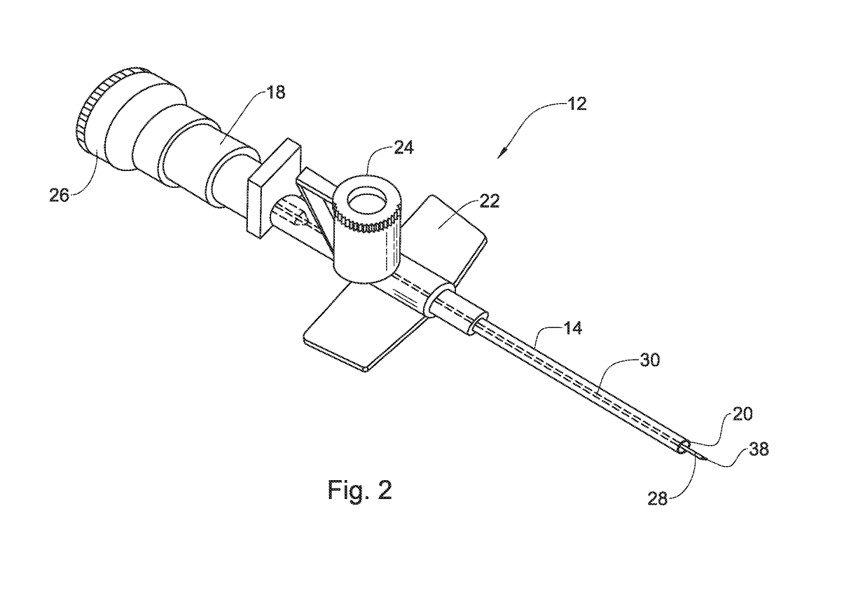

In operation, the trocar 28 is inserted into the first cannula 12, as shown in FIG. 2. The outer diameter of the hollow tubular portion 30 of the trocar 28 is selected to be less than the inner diameter of the first cannula 12 and specifically the distal portion of the first cannula 14, so that the trocar 28 can slide within the distal portion of the first cannula 14. The length of the hollow tubular portion 30 of the trocar 28 is selected to allow the tip 38 of the trocar 28 to extend beyond the tip 20 of the distal portion of the first cannula 14 when inserted within the first cannula 12.

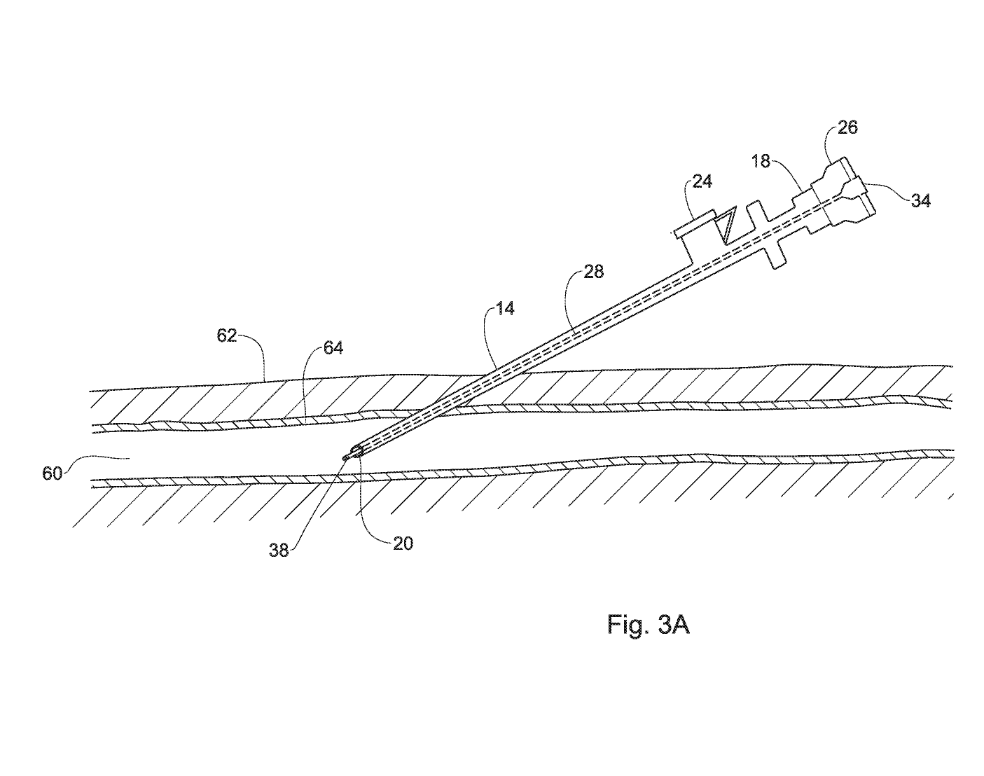

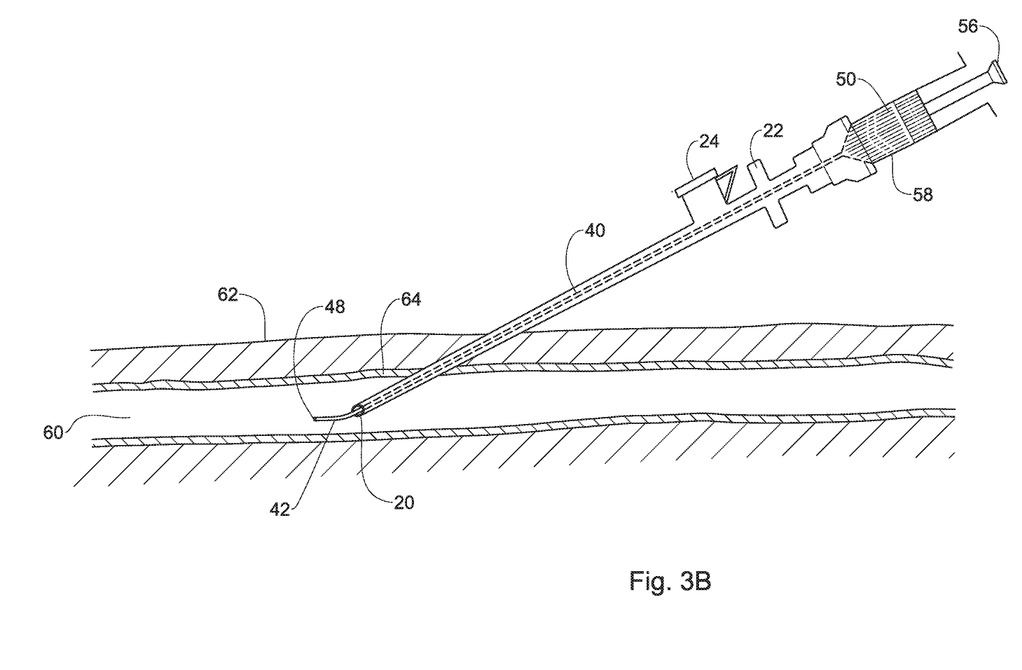

FIGS. 3A and 3B show an exemplary use of the cannula assembly 10 for delivering or withdrawing fluid (such as blood or medicament) to or from any body cavity such as a blood vessel 60. After insertion of the trocar 28 into the first cannula 12 and into the distal portion 14, the tip 38 of the trocar 28 is used to pierce the skin surface 62. The tip 38, when located, within the first cannula 12 is pushed forward until the tip 38 of the trocar 28 pierces the blood vessel wall 64 and the tip 38 of the trocar and the tip 20 of the first cannula are positioned within the blood vessel 60. Insertion of the tip 20 of the first cannula inside the blood vessel 60 is evidenced by the presence of blood in the flashback chamber 18. Once the tip 20 of the first cannula is positioned in the blood vessel, the trocar 28 is removed from the first cannula 12. The first cannula 12 can then be affixed to the skin surface 62, for example, by taping the wings 22 (not seen in FIG. 3A) to the skin surface 62. The cannula extends into a vein for a predetermined distance chosen so as to avoid collapse of the blood vessel, particularly where the blood vessel is vein, which may result from interaction with valves within the vein and resulting flow-related, e.g. venturi effect.

As shown in FIG. 3B, the flexible second cannula 40 is coupled to the syringe 50 having a plunger 56 in its depressed position. The flexible second cannula 40 is then inserted into the first cannula 12 until the tip 48 of the second cannula 40 extends beyond the tip 20 of the first cannula 12 in the blood vessel 60. The plunger 56 can then be pulled back causing blood 58 to fill the barrel of the syringe 50. As may be appreciated, blood withdrawal using a syringe is one of a number of blood-withdrawal means. Another example is through the use of a blood collection device, e.g. blood collection tube with pre-formed vacuum.

After removal of blood, the syringe 50 and the flexible second cannula 40 are removed from the first cannula 12. The first cannula device 12 remains affixed to the skin surface 62. The Luer fitting 26 is stoppered with a stopper (not shown) to prevent loss of blood from the blood vessel 60 or entry of pathogens into the device.

At any time when another blood sample 58 is to be withdrawn, an additional fresh flexible cannula 40 can be mounted onto a new and fresh syringe 50. The stopper (not shown) is removed and the flexible second cannula 40 is inserted through the first cannula 12 into the blood vessel 60, as shown in FIG. 3B, and the process repeated. Repeated withdrawals of blood 58 can thus be performed without piercing the skin repeatedly with the trocar 28 and without the need to change the first cannula 12.

The flexible cannula can, once inserted, be used to infuse fluid or medication into a vessel or to withdraw fluid or blood samples for testing and analysis.

This disclosure has been illustrated with reference to particular embodiments, it should be understood that these embodiments are merely illustrative of the principles and practice as generally described above, and that various modifications will be apparent to persons of ordinary skill in the art. It is therefore intended that all such modifications are to be included within the spirit and scope of the invention as defined by the following claims.

It must be noted that, as used in this specification and the appended claims, the singular forms “a”, “an” and “the” include plural referents unless the content clearly dictates otherwise.

The terms “comprises”, “comprising”, “includes”, “including”, “having” mean “including but not limited to”. These terms encompasses the terms “consisting of” and “consisting essentially of”.

1. A cannula assembly, comprising:
a first cannula device having a first length and first inner diameter and having at least a flexible distal portion;
a trocar element having a second length and second diameter and having a sharp distal portion capable of puncturing through skin and into a blood vessel of a patient; the trocar element being sized such that the second diameter permits it to be inserted within the first cannula and has the second length permits its distal end once inserted, to extend beyond the flexible distal portion of the first cannula; and
at least one flexible second cannula is configured for insertion through the first cannula, the second flexible cannula sized to extend beyond the distal portion of the first cannula device, for a predetermined length within the blood vessel of the patient.

2. A cannula assembly according to claim 1, wherein the predetermined length of the second cannula is chosen to avoid collapse of a patient's blood vessel in which the cannula is inserted.

3. A cannula assembly according to claim 1, wherein the second cannula has a proximal end configured for coupling to a syringe, a blood collection device or an intravenous solution adapter.

4. A method for infusing a fluid into or withdrawing a fluid from a blood vessel of a patient, comprising:
providing a cannula assembly comprising (i) a first cannula device having a first length and first inner diameter and having at least a flexible distal portion; (ii) a trocar element having a second length and second diameter and having a sharp distal portion capable of puncturing through skin and into a blood vessel of a patient; and (iii) at least one flexible second cannula;
inserting the first cannula and the trocar within the first cannula into a blood vessel of a patient by puncturing through skin into the blood vessel of the patient;
withdrawing the trocar from the first cannula; and
inserting the second cannula into the first cannula such that the distal end of the second cannula extends a distance in the blood vessel of the patient beyond the distal end of the first cannula.

5. The method according to claim 4, further comprising connecting a syringe or a blood collection device to a proximal end of the first cannula and withdrawing blood from the patient.

6. The method according to claim 4, further comprising connecting a liquid dispenser to a proximal end of the first cannula and infusing a liquid into the blood vessel of the patient.

7. The method according to claim 4, wherein the second cannula is withdrawn from the first cannula after use leaving the first cannula in the blood vessel of the patient and capable of accepting another second cannula for repeated access to the blood vessel.

8. The method according to claim 4, wherein the trocar element being sized such that the second diameter permits it to be inserted within the first cannula and has the second length permits its distal end once inserted, to extend beyond the flexible distal portion of the first cannula

9. The method according to claim 4, wherein the second cannula being configured for insertion through the first cannula and being sized to extend beyond the distal portion of the first cannula device, for a predetermined length within the blood vessel of the patient.
